(19) United States
(12) Patent Application Publication
(10) Pub. No.: US 2016/0083331 A1
(43) Pub. Date: Mar. 24, 2016

(54) POLYFUNCTIONAL (METH)ACRYLATE, AND METHOD FOR PRODUCING SAME

(71) Applicant: DAICEL CORPORATION, Osaka-shi, Osaka (JP)

(72) Inventors: Nobuhiko HARADA, Himeji-shi (JP); Ichiro TAKASE, Himeji-shi (JP)

(73) Assignee: DAICEL CORPORATION, Osaka-shi, Osaka (JP)

(21) Appl. No.: 14/891,931

(22) PCT Filed: Jun. 11, 2014
(86) PCT No.: PCT/JP2014/065489

§ 371 Date: Nov. 17, 2015

(30) Foreign Application Priority Data
Jun. 21, 2013 (JP) 2013-130877

(51) Int. Cl. C07C 69/54; C09D 4/00; C08F 220/18; C07C 67/26; C08F 222/14

(52) U.S. Cl. CPC C07C 69/54; C07C 67/26; C08F 222/14; C08F 220/18; C09D 4/00; C07C 2103/68

(57) ABSTRACT

Provided are: a multifunctional (meth)acrylate compound that has excellent curability and can form a polymer or cured product having hardness and durability at excellent levels; and a method for easily and selectively producing the multifunctional (meth)acrylate compound. The multifunctional (meth)acrylate compound according to the present invention is represented by Formula (1) below. In the formula, Ring Z¹ represents a tricyclo[5.2.1.0^{2,6}]decane ring, R is selected from a hydrogen atom and a methyl group, and n¹ and n² meet a condition that n¹ is 2 or 3 and n² is 1, or a condition that n¹ is 3 or 4 and n² is 0.

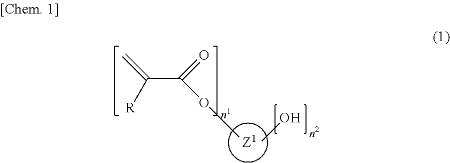

### TECHNICAL FIELD

The present invention relates to a novel compound containing two or more (meth)acryloyl groups on a bridged carbon ring, and to a method for producing the novel compound. The novel compound, when polymerized, gives a polymer that is useful as a glass-substitute material. The present application claims priority to Japanese Patent Application No. 2013-130877 filed to Japan on Jun. 21, 2013, the entire contents of which are incorporated herein by reference.

### BACKGROUND ART

Active-energy-ray-polymerizable compounds include a variety of (meth)acrylates and are often used in or for waveguides (e.g., optical waveguides), hybrid boards, optical fibers, stress-relaxation adhesives, encapsulating agents (sealing agents), underfill materials, ink-jet inks, color filters, nanoimprinting, flexible substrates, and any other uses.

Of the active-energy-ray-polymerizable compounds, multifunctional (meth)acrylates each containing a bridged carbon ring skeleton, in particular, have high hardness and excellent durability as compared with glass and have light weights about half the weight of glass. These multifunctional (meth)acrylates can be subjected to injection molding, thereby have high degree of freedom in shape, can form two or more members or components integrally, and contribute to better vehicle design and better productivity. The multifunctional (meth)acrylates receive attention as glass-substitute materials and are expected to be used typically in or for automobile grazing such as windshields; high-hardness hard coat films for portable communication devices and portable information devices; and optical sheets.

Among such multifunctional (meth)acrylates each containing a bridged carbon ring skeleton, for example, tricyclodecanediol di(meth)acrylate and tricyclodecanedimethanol di(meth)acrylate are known to be capable of forming cured products that have hardness and durability at excellent levels (Patent Literature (PTL) 1). The properties of them are, however, still insufficient in some uses. There is a need of providing a compound that can form a cured product having hardness and durability at still more excellent levels. In addition, it is difficult to prepare tricyclodecanediol di(meth)acrylate synthetically. In addition, the introduction rate of a functional group into the compound so as to impart hardness and durability to the compound is low, resulting in impracticality. Accordingly, under present circumstances, there is found no monomer that can be efficiently produced by an easy method and, when polymerized, can form a cured product having hardness and durability at excellent levels.

### CITATION LIST

### Patent Literature

PTL 1: Japanese Unexamined Patent Application Publication (JP-A) No. 2006-315960

### SUMMARY OF INVENTION

### Technical Problem

Accordingly, the present invention has an object to provide a multifunctional (meth)acrylate compound that has excellent curability and can form a polymer or cured product having hardness and durability at excellent levels.

The present invention has another object to provide a method for easily and selectively producing the multifunctional (meth)acrylate compound.

The present invention has yet another object to provide a mixture that includes the multifunctional (meth)acrylate compound, has excellent curability, and can form a cured product having hardness and durability at excellent levels.

### Solution to Problem

After intensive investigations to achieve the objects, the inventors of the present invention have found that a compound containing two to four (meth)acryloyl groups on a tricyclo[5.2.1.0^{2,6}]decane ring can be selectively produced by an easy method as follows. The method employs, as a raw material, a compound corresponding to a tricyclo[5.2.1.0^{2,6}]decane compound, except for containing one or two carbon-carbon double bonds respectively instead of one or two carbon-carbon single bonds constituting the tricyclo[5.2.1.0^{2,6}]decane. In the method, the carbon-carbon double bond or bonds are epoxidized to form epoxy group or groups, and the epoxy group or groups are further (meth)acrylated to give the target compound. The inventors have also found that the compound obtained by the production method, and a specific mixture, when polymerized, can form cured products that have hardness and durability at excellent levels, where the mixture includes a specific proportion of a compound containing one or two (meth)acryloyl groups on a tricyclo[5.2.1.0^{2,6}]decane ring. The present invention has been made based on these findings.

Specifically, the present invention provides a multifunctional (meth)acrylate compound represented by Formula (1):

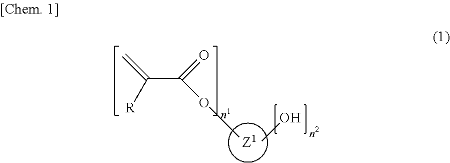

where Ring Z¹ represents a tricyclo[5.2.1.0^{2,6}]decane ring; R is, independently in each occurrence, selected from a hydrogen atom and a methyl group; and n¹ and n² meet a condition that n¹ is 2 or 3 and n² is 1, or a condition that n¹ is 3 or 4 and n² is 0.

The present invention provides, in another embodiment, a mixture including a multifunctional (meth)acrylate compound represented by Formula (1):

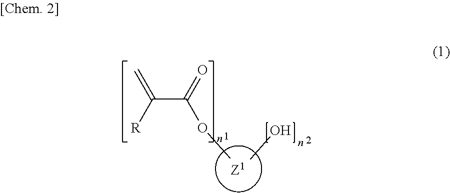

where Ring Z¹ represents a tricyclo[5.2.1.0^{2,6}]decane ring; R is, independently in each occurrence, selected from a hydrogen atom and a methyl group; and n¹ and n² meet a condition that n¹ is 2 or 3 and n² is 1, or a condition that n¹ is 3 or 4 and n² is 0.

The mixture may be obtained by allowing a compound represented by Formula (2) or a compound represented by Formula (3) to react with (meth)acrylic acid. Formulae (2) and (3) are expressed as follows:

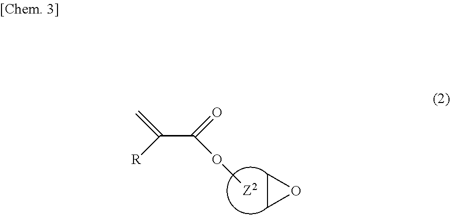

where Ring Z² is a ring corresponding to a tricyclo[5.2.1.0^{2,6}]decane ring, except for having one epoxy group, where the epoxy group contains one oxygen atom bonded in a triangular arrangement to two adjacent carbon atoms constituting the tricyclo[5.2.1.0^{2,6}]decane ring; and R is selected from a hydrogen atom and a methyl group,

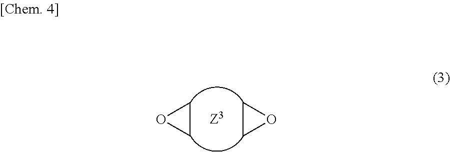

where Ring Z³ is a ring corresponding to a tricyclo[5.2.1.0^{2,6}]decane ring, except for having two epoxy groups, where the two epoxy groups each independently contain one oxygen atom bonded in a triangular arrangement to two adjacent carbon atoms constituting the tricyclo[5.2.1.0^{2,6}]decane ring.

The present invention provides, in yet another embodiment, a mixture of (meth)acrylate compounds including a di(meth)acrylate compound represented by Formula (1′) and a mono(meth)acrylate compound represented by Formula (4). The mixture includes the di(meth)acrylate compound in a proportion of equal to or more than 10 percent by weight of all the (meth)acrylate compounds contained in the mixture. Formulae (1′) and (4) are expressed as follows:

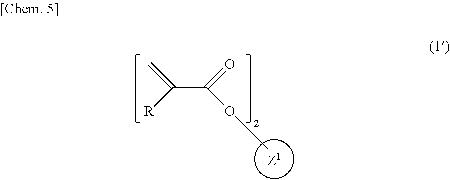

where Ring Z¹ represents a tricyclo[5.2.1.0^{2,6}]decane ring; and R is, independently in each occurrence, selected from a hydrogen atom and a methyl group,

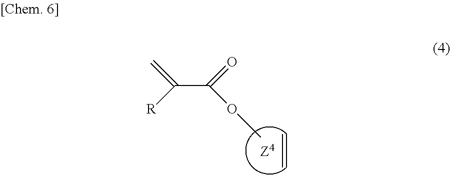

where Ring Z⁴ is a ring corresponding to a tricyclo[5.2.1.0^{2,6}]decane ring, except for containing one carbon-carbon double bond instead of one of carbon-carbon single bonds constituting the tricyclo[5.2.1.0^{2,6}]decane ring; and R is selected from a hydrogen atom and a methyl group.

The above-mentioned mixture may be obtained by allowing 1 mole of tricyclo[5.2.1.0^{2,6}]deca-3,8-diene to react with 1.1 moles or more of (meth)acrylic acid in the presence of a Lewis acid catalyst.

In the mixture, the Lewis acid catalyst may be boron trifluoride-diethyl ether complex.

The present invention further provides, in another embodiment, a method for producing a multifunctional (meth)acrylate compound, where the method includes allowing a compound represented by Formula (2) or a compound represented by Formula (3) to react with (meth)acrylic acid to yield a multifunctional (meth)acrylate compound represented by Formula (1). Formulae (2), (3), and (1) are expressed as follows:

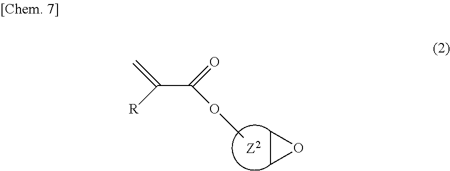

where Ring Z² is a ring corresponding to a tricyclo[5.2.1.0^{2,6}]decane ring, except for having one epoxy group, where the epoxy group contains one oxygen atom bonded in a triangular arrangement to two adjacent carbon atoms constituting the tricyclo[5.2.1.0^{2,6}]decane ring; and R is selected from a hydrogen atom and a methyl group,

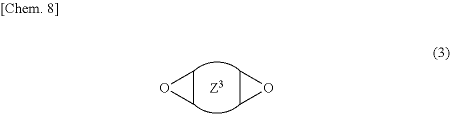

where Ring Z³ is a ring corresponding to a tricyclo[5.2.1.0^{2,6}]decane ring, except for having two epoxy groups, where the two epoxy groups each independently contain one oxygen atom bonded in a triangular arrangement to two adjacent carbon atoms constituting the tricyclo[5.2.1.0^{2,6}]decane ring,

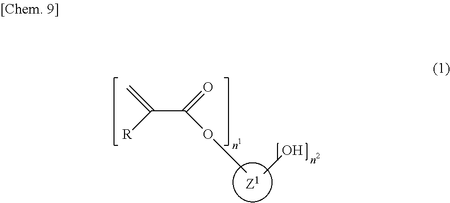

where Ring Z¹ represents a tricyclo[5.2.1.0^{2,6}]decane ring; n¹ and n² meet a condition that n¹ is 2 or 3 and n² is 1, or a condition that n¹ is 3 or 4 and n² is 0; and R is as defined above.

In the method for producing a multifunctional (meth)acrylate compound, a compound represented by Formula (4) may be allowed to react with a peracid to yield a compound represented by Formula (2), and the obtained compound represented by Formula (2) may be allowed to react with (meth)acrylic acid. Formulae (2) and (4) are expressed as follows:

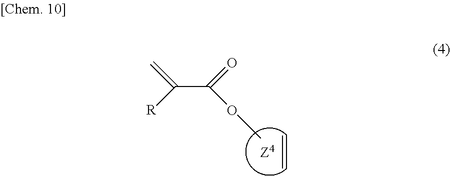

where Ring Z⁴ is a ring corresponding to a tricyclo[5.2.1.0^{2,6}]decane ring, except for containing one carbon-carbon double bond instead of one of carbon-carbon single bonds constituting the tricyclo[5.2.1.0^{2,6}]decane ring; and R is selected from a hydrogen atom and a methyl group,

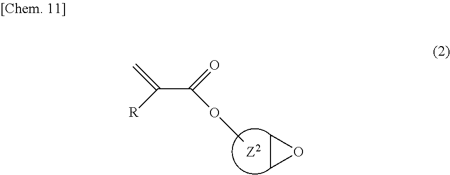

where Ring Z² is a ring corresponding to a tricyclo[5.2.1.0^{2,6}]decane ring, except for having one epoxy group, where the epoxy group contains one oxygen atom bonded in a triangular arrangement to two adjacent carbon atoms constituting the tricyclo[5.2.1.0^{2,6}]decane ring; and R is as defined above.

In the method for producing a multifunctional (meth)acrylate compound, a compound represented by Formula (5) may be allowed to react with a peracid to yield a compound represented by Formula (3), and the obtained compound represented by Formula (3) may be allowed to react with (meth)acrylic acid. Formulae (5) and (3) are expressed as follows:

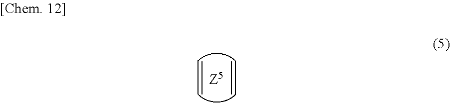

where Ring Z⁵ is a ring corresponding to a tricyclo[5.2.1.0^{2,6}]decane ring, except for containing two carbon-carbon double bonds instead of two carbon-carbon single bonds constituting the tricyclo[5.2.1.0^{2,6}]decane ring,

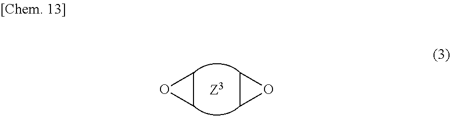

where Ring Z³ is a ring corresponding to a tricyclo[5.2.1.0^{2,6}]decane ring, except for having two epoxy groups, where the two epoxy groups each independently contain one oxygen atom bonded in a triangular arrangement to two adjacent carbon atoms constituting the tricyclo[5.2.1.0^{2,6}]decane ring.

The present invention further provides a polymer including a monomer unit derived from the multifunctional (meth)acrylate compound.

The present invention also provides a high-hardness hard-coating agent including the multifunctional (meth)acrylate compound or any of the mixtures.

In addition, the present invention provides a cured product of the high-hardness hard-coating agent.

### Advantageous Effects of Invention

The multifunctional (meth)acrylate compounds according to the present invention have the configurations, offer excellent curability (polymerizability), and can form polymers or cured products having hardness and elastic modulus at high levels and having excellent strength. The multifunctional (meth)acrylate compounds are therefore useful typically as or for glass-substitute materials.

Of the multifunctional (meth)acrylate compounds according to the present invention, hydroxy-containing compounds each have a hydroxy group that can be easily converted into a specific chemical group. The hydroxy-containing compounds, when undergone the conversion, are usable as raw materials (i.e., functional monomers) to form resins (i.e., functional resins) having specific excellent function or functions. For example, such a hydroxy-containing compound, when its hydroxy group is allowed to react with an isocyanate compound, gives a compound having a urethane bond. This compound, when polymerized, can form a polymer or cured product having flexibility and substrate adhesion at excellent levels. Thus, the multifunctional (meth)acrylate compounds according to the present invention are useful also as precursors for functional monomers.

### DESCRIPTION OF EMBODIMENTS

The multifunctional (meth)acrylate compounds according to the present invention are represented by Formula (1):

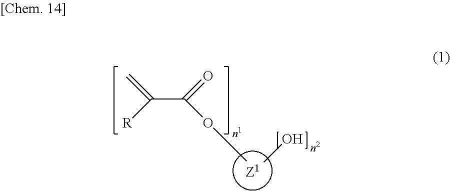

where Ring Z¹ represents a tricyclo[5.2.1.0^{2,6}]decane ring; R is, independently in each occurrence, selected from a hydrogen atom and a methyl group; and n¹ and n² meet a condition that n¹ is 2 or 3 and n² is 1, or a condition that n¹ is 3 or 4 and n² is 0.

Examples of the multifunctional (meth)acrylate compounds according to the present invention include, but are not limited to, compounds represented by Formulae (1-a) to (1-k) (including stereoisomers such as endo isomers and exo isomers). In the formulae, multiple occurrences of R may be identical or different.

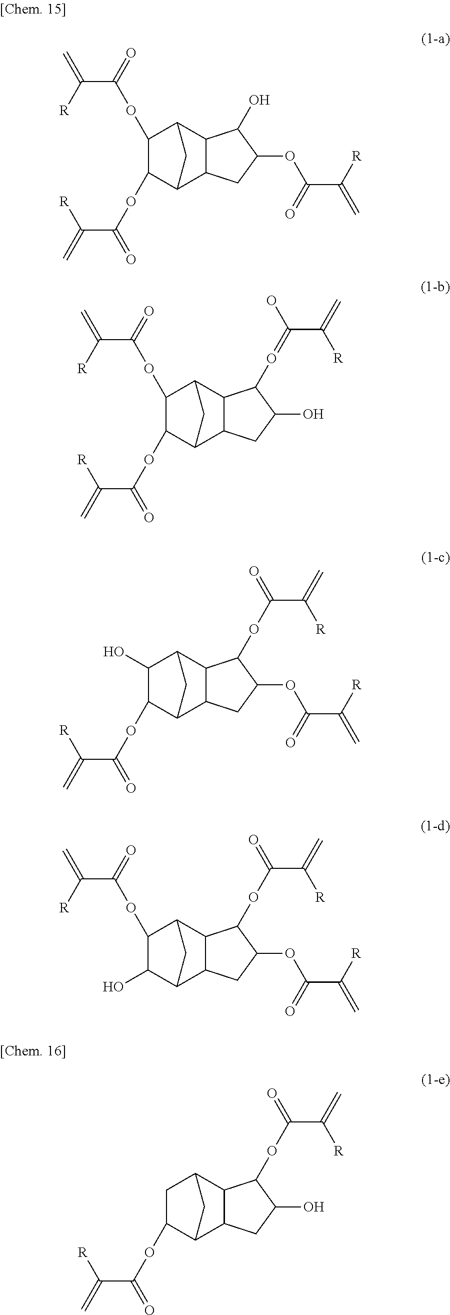

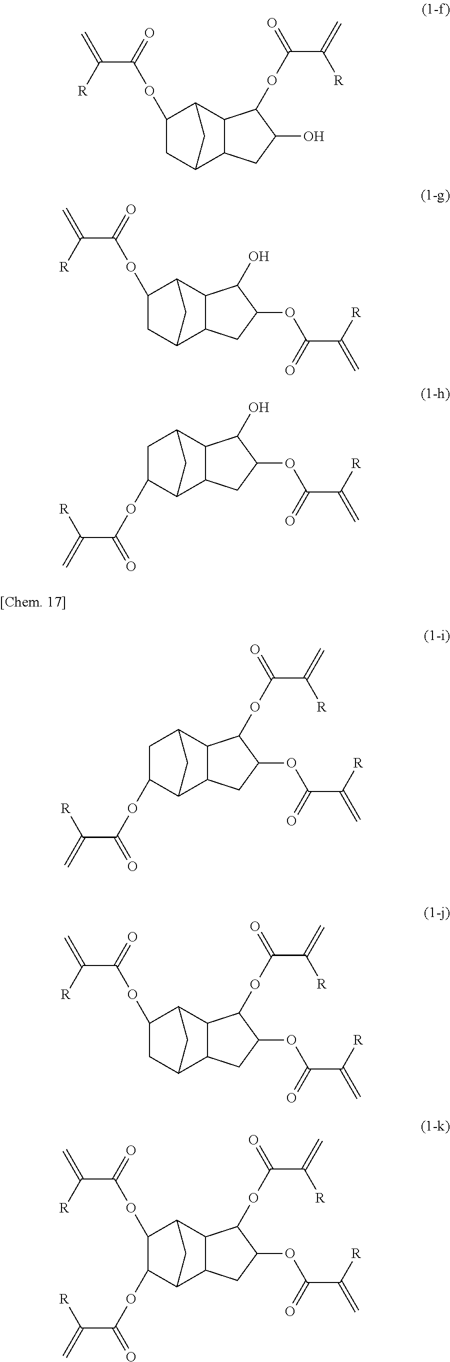

The multifunctional (meth)acrylate compounds according to the present invention may each be produced by allowing a compound represented by Formula (2) or a compound represented by Formula (3) to react with (meth)acrylic acid to thereby (meth)acrylate the epoxy group or groups. Formulae (2) and (3) are expressed as follows:

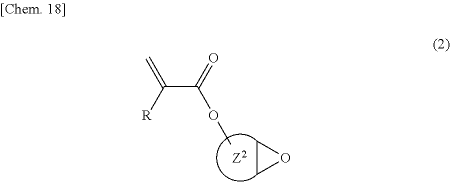

where Ring Z² is a ring corresponding to a tricyclo[5.2.1.0^{2,6}]decane ring, except for having one epoxy group, where the epoxy group contains one oxygen atom bonded in a triangular arrangement to two adjacent carbon atoms constituting the tricyclo[5.2.1.0^{2,6}]decane ring; and R is selected from a hydrogen atom and a methyl group,

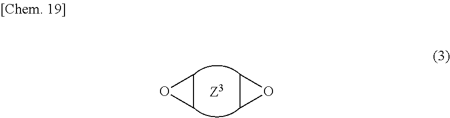

where Ring Z³ is a ring corresponding to a tricyclo[5.2.1.0^{2,6}]decane ring, except for having two epoxy groups, where the two epoxy groups each independently contain one oxygen atom bonded in a triangular arrangement to two adjacent carbon atoms constituting the tricyclo[5.2.1.0^{2,6}]decane ring.

The Ring Z² is a ring corresponding to a tricyclo[5.2.1.0^{2,6}]decane ring, except for having one epoxy group, where the epoxy group contains one oxygen atom bonded in a triangular arrangement to two adjacent carbon atoms constituting the tricyclo[5.2.1.0^{2,6}]decane ring. Examples of Ring Z² include, but are not limited to, rings (including stereoisomers) represented by Formulae (2-1) and (2-2):

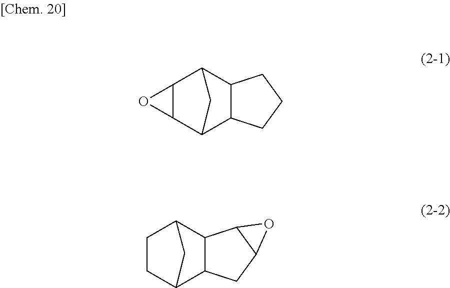 [SMILES: C1CC2C(C1)C1CC2C2OC12 ; C1CC2CC1C1CC3OC3C21]

The Ring Z³ is a ring corresponding to a tricyclo[5.2.1.0^{2,6}]decane ring, except for having two epoxy groups, where the two epoxy groups each independently contain one oxygen atom bonded in a triangular arrangement to two adjacent carbon atoms constituting the tricyclo[5.2.1.0^{2,6}]decane ring. A non-limiting example of Ring Z³ is a ring (including stereoisomers) represented by Formula (3-1):

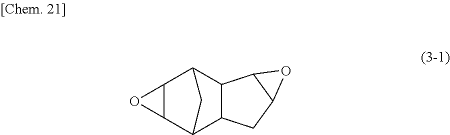 [SMILES: C1C2OC2C2C1C1CC2C2OC12]

Of the compounds represented by Formula (1), a compound in which n¹ is 2 and n² is 1 (a compound represented by Formula (1-I) below) may be produced by allowing 1 mole of the compound represented by Formula (2) to react with about 1 to about 10 moles of (meth)acrylic acid. Alternatively, the compound represented by Formula (1-I) may be produced by blending the compound represented by Formula (2) and an excess amount of (meth)acrylic acid and allowing them to react with each other for about 1 to about 20 hours.

Of the compounds represented by Formula (1), a compound in which n¹ is 3 and n² is 0 (a compound represented by Formula (1-II) below) may be produced by allowing 1 mole of the compound represented by Formula (2) to react with about 2 to about 10 moles of (meth)acrylic acid. Alternatively, the compound represented by Formula (1-II) may be produced by blending the compound represented by Formula (2) and an excess amount of (meth)acrylic acid and allowing them to react with each other for about 1 to about 20 hours.

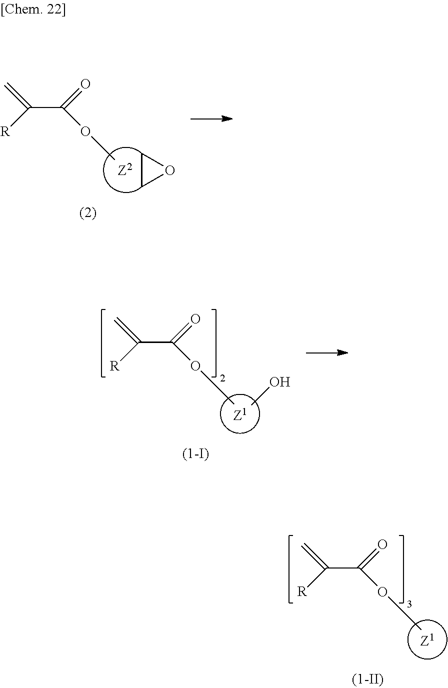

Of the compounds represented by Formula (1), a compound in which n¹ is 3 and n² is 1 (a compound represented by Formula (1-III) below) may be produced by allowing 1 mole of the compound represented by Formula (3) to react with about 1 to about 20 moles of (meth)acrylic acid. Alternatively, the compound represented by Formula (1-III) may be produced by blending the compound represented by Formula (3) and an excess amount of (meth)acrylic acid and allowing them to react with each other for about 1 to about 20 hours.

Of the compounds represented by Formula (1), a compound in which n¹ is 4 and n² is 0 (a compound represented by Formula (1-IV) below) may be produced by allowing 1 mole of the compound represented by Formula (3) to react with about 2 to about 10 moles of (meth)acrylic acid. Alternatively, the compound represented by Formula (1-IV) may be produced by blending the compound represented by Formula (3) and an excess amount of (meth)acrylic acid, and allowing them to react with each other for about 1 to about 20 hours.

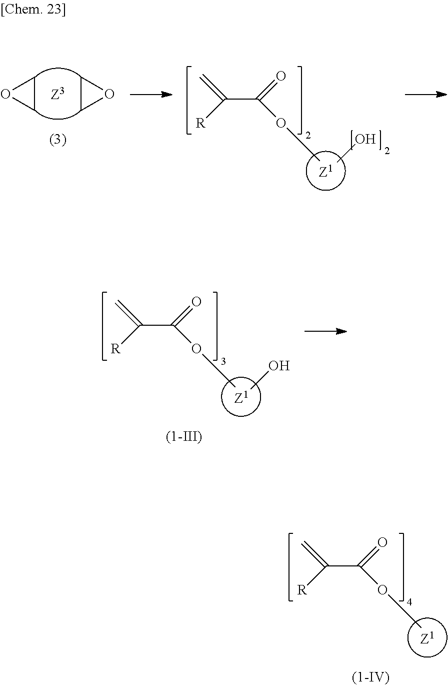

The reactions are preferably performed in the presence of a polymerization inhibitor. Examples of the polymerization inhibitor include, but are not limited to, hydroquinone, hydroquinone monomethyl ether, phenothiazine, 4,4′-thiobis(6-t-butyl-m-cresol), 4,4′-butylidenebis(6-t-butyl-m-cresol), 1,1,3-tris(5-t-butyl-4-hydroxy-2-methylphenyl)butane, p-methoxyphenol, and 6-t-butyl-2,4-xylenol. Each of them may be used alone or in combination. The polymerization inhibitor(s) may be used in an amount of typically about 0.001 to about 0.5 mole, and preferably 0.005 to 0.1 mole, per 1 mole of the multifunctional (meth)acrylate compound represented by Formula (1) to be formed. The polymerization inhibitor, if used in an amount less than the range, may fail to sufficiently effectively inhibit polymerization. In contrast, the polymerization inhibitor, if used in an amount greater than the range, may adversely affect properties of the product.

The coexistence of a component including molecular oxygen in the reaction system may also restrain a polymerization reaction. Examples of the component include, but are not limited to, air; and oxygen diluted typically with nitrogen. The above-mentioned reactions are preferably performed in a molecular-oxygen-containing gas atmosphere. The oxygen concentration may be selected as appropriate in consideration of safety.

The reactions are preferably performed at a temperature of 130° C. or lower (e.g., 50° C. to 130° C.). The reaction, if performed at a temperature lower than the range, may fail to proceed at a sufficient reaction rate. In contrast, the reaction, if performed at a temperature higher than the range, may cause a heat-induced radical polymerization reaction to proceed. This may lead to crosslinking of the double bond moiety or moieties, resulting in gelation.

The reactions are generally performed in the presence of a base. Examples of the base include organic bases and inorganic bases. Examples of the organic bases include, but are not limited to, tertiary amines such as triethylamine and N-methylpiperidine; nitrogen-containing heteroaromatic compounds such as pyridine; alkali metal alkoxides such as sodium methoxide; and organic acid alkali metal salts such as sodium acetate and potassium (meth)acrylate. Examples of the inorganic bases include, but are not limited to, alkali metals (elementary alkali metals) such as sodium; alkali metal hydroxides such as sodium hydroxide, alkali metal carbonates such as sodium carbonate, and alkali metal hydrogencarbonates such as sodium hydrogencarbonate. Each of them may be used alone or in combination. In the present invention, the base is preferably selected from elementary alkali metals and organic acid alkali metal salts. The base may be used in an amount of typically about 0.001 to about 0.5 mole, and preferably 0.01 to 0.4 mole, per 1 mole of the compound represented by Formula (2) or the compound represented by Formula (3). The base may also be used in large excess.

The reactions may be performed in the presence of a solvent. The solvent is not limited, as long as not adversely affecting the reaction progress, and is exemplified by aromatic hydrocarbons such as toluene and benzene; aliphatic hydrocarbons such as hexane; alicyclic hydrocarbons such as cyclohexane; and esters such as ethyl acetate. Each of them may be used alone or in combination.

The reactions may be performed according to any system such as batch system, semi-batch system, or continuous system. After the completion of the reaction, a reaction product may be separated/purified by a separation/purification procedure such as filtration, concentration, distillation, extraction, crystallization, recrystallization, adsorption, and column chromatography, or a procedure as any combination of them.

The compound represented by Formula (2) may be produced typically by allowing a compound represented by Formula (4) to react with a peracid to epoxidize the carbon-carbon double bond of the compound represented by Formula (4). Formula (4) is expressed as follows:

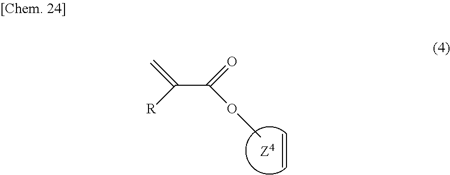

where Ring Z⁴ is a ring corresponding to a tricyclo[5.2.1.0^{2,6}]decane ring, except for containing one carbon-carbon double bond instead of one of carbon-carbon single bonds constituting the tricyclo[5.2.1.0^{2,6}]decane ring; and R is selected from a hydrogen atom and a methyl group.

The compound represented by Formula (3) may be produced typically by allowing a compound represented by Formula (5) to react with a peracid to epoxidize the carbon-carbon double bonds of the compound represented by Formula (5). Formula (5) is expressed as follows:

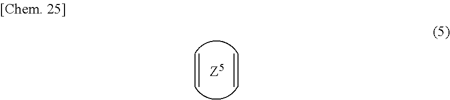

where Ring Z⁵ is a ring corresponding to a tricyclo[5.2.1.0^{2,6}]decane ring, except for containing two carbon-carbon double bonds instead of two carbon-carbon single bonds constituting the tricyclo[5.2.1.0^{2,6}]decane ring.

The Ring Z⁴ is a ring corresponding to a tricyclo[5.2.1.0^{2,6}]decane ring, except for containing one carbon-carbon double bond instead of one of carbon-carbon single bonds constituting the tricyclo[5.2.1.0^{2,6}]decane ring. Examples of Ring Z⁴ include, but are not limited to, rings (including stereoisomers) represented by Formulae (4-1) and (4-2):

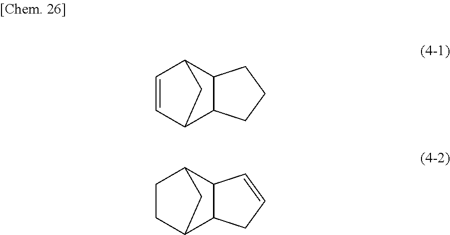

The Ring Z⁵ is a ring corresponding to a tricyclo[5.2.1.0^{2,6}]decane ring, except for containing two carbon-carbon double bonds instead of two carbon-carbon single bonds constituting the tricyclo[5.2.1.0^{2,6}]decane ring. Examples of Ring Z⁵ include a ring (including stereoisomers) represented by Formula (5-1):

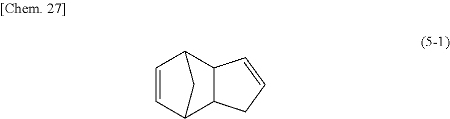

Examples of the peracid include, but are not limited to, performic acid, peracetic acid, perbenzoic acid, m-chloroperbenzoic acid, and trifluoroperacetic acid. Each of them may be used alone or in combination.

The peracid may be used in an amount of typically about 0.5 to about 6 moles, and preferably 1 to 3 moles, per 1 mole of the carbon-carbon double bond(s) of the compound represented by Formula (4) or the compound represented by Formula (5).

The above-mentioned reactions may be performed in the presence of a solvent. The solvent is not limited, as long as not adversely affecting the reaction progress, and is exemplified by aromatic hydrocarbons such as toluene and benzene; aliphatic hydrocarbons such as hexane; alicyclic hydrocarbons such as cyclohexane; and esters such as ethyl acetate. Each of them may be used alone or in combination.

The reaction atmosphere is not limited, as long as not adversely affecting the reaction, and may be selected from any atmospheres such as nitrogen atmosphere and argon atmosphere. The reactions may be performed at a temperature of typically about −20° C. to about 80° C., and preferably 0° C. to 60° C., for a reaction time of typically about 1 to about 10 hours.

The reactions may be performed according to any system such as batch system, semi-batch system, or continuous system. After the completion of the reaction, a reaction product may be separated/purified by a separation/purification procedure such as filtration, concentration, distillation, extraction, crystallization, recrystallization, adsorption, or column chromatography, or a procedure as any combination of them.

The compound represented by Formula (4) may be produced by allowing the compound represented by Formula (5) to react with (meth)acrylic acid.

The (meth)acrylic acid may be used in an amount of typically about 0.5 to about 20 moles, and preferably 1 to 10 moles, per 1 mole of the compound represented by Formula (5).

The above-mentioned reaction is preferably performed in the presence of a catalyst. Examples of the catalyst include, but are not limited to, catalysts that may be generally usable in Friedel-Crafts reactions, including Lewis acid catalysts such as anhydrous aluminum chloride, anhydrous aluminum bromide, anhydrous iron chloride, titanium tetrachloride, tin tetrachloride, zinc chloride, boron trifluoride-diethyl ether complex, anhydrous boron trioxide, and concentrated sulfuric acid; and sulfonic acids such as trifluoromethanesulfonic acid. Each of them may be used alone or in combination.

The catalyst(s) may be used in an amount of typically about 0.001 to about 0.5 mole, and preferably 0.01 to 0.1 mole, per 1 mole of the compound represented by Formula (5).

The reaction is preferably performed in the presence of a polymerization inhibitor. The polymerization inhibitor may be exemplified as above. The polymerization inhibitor may be used in an amount of typically about 0.001 to about 0.5 mole, and preferably 0.005 to 0.1 mole, per 1 mole of the compound represented by Formula (4) to be formed.

The reaction atmosphere is not limited, as long as not adversely affecting the reaction, and may be selected from any atmospheres such as nitrogen atmosphere and argon atmosphere. The reaction may be performed at a temperature of typically about 30° C. to about 130° C., and preferably 40° C. to 120° C., for a reaction time of typically about 0.5 to about 10 hours.

The reaction may be performed according to any system such as batch system, semi-batch system, or continuous system. After the completion of the reaction, a reaction product may be separated/purified by a separation/purification procedure such as filtration, concentration, distillation, extraction, crystallization, recrystallization, adsorption, or column chromatography, or a procedure as any combination of them.

The production method can give the multifunctional (meth)acrylate compound represented by Formula (1) efficiently (in a yield of typically equal to or more than 80%).

The production method gives one selected from, or a mixture of two or more selected from, all stereoisomers and regioisomers in the multifunctional (meth)acrylate compound represented by Formula (1).

The multifunctional (meth)acrylate compounds represented by Formula (1), which may be obtained by the production method, offer excellent polymerizability and, when polymerized alone or in combination with another polymerizable compound, can form a polymer. Examples of the other polymerizable compound include, but are not limited to, (meth)acrylate compounds excluding the multifunctional (meth)acrylate compounds represented by Formula (1). The polymerization may be performed by a common technique such as solution polymerization, bulk polymerization, suspension polymerization, bulk and suspension polymerization, emulsion polymerization, and any other polymerization techniques. The polymerization may be performed in the presence typically of a polymerization initiator as added.

The mixtures according to the present invention each include at least one of the multifunctional (meth)acrylate compounds represented by Formula (1) (including stereoisomers and regioisomers). The mixtures preferably each include two or more different ones selected from the compounds represented by Formula (1).

In particular, of the mixtures according to the present invention, preferred is a mixture containing both a hydroxy di(meth)acrylate compound of Formula (1) in which n¹ is 2 and n² is 1, and a tri(meth)acrylate compound of Formula (1) in which n¹ is 3 and n² is 0. In this mixture, the total content of the hydroxy di(meth)acrylate compound and the tri(meth)acrylate compound may be typically equal to or more than 50 percent by weight (preferably equal to or more than 70 percent by weight, particularly preferably equal to or more than 80 percent by weigh, and most preferably from 90 percent by weight to 100 percent by weight) of the total weight of the mixture. The ratio (weight ratio) of the hydroxy di(meth)acrylate compound to the tri(meth)acrylate compound is typically from 60:40 to 99.5:0.5, preferably from 70:30 to 99:1, particularly preferably from 80:20 to 98.5:1.5, and most preferably from 90:10 to 98:2.

The mixtures according to the present invention further include a mixture of (meth)acrylate compounds as follows. The mixture of (meth)acrylate compounds includes a di(meth)acrylate compound represented by Formula (1′) and a mono(meth)acrylate compound represented by Formula (4). The mixture includes the di(meth)acrylate compound in a proportion of equal to or more than 10 percent by weight (preferably equal to or more than 15 percent by weight, particularly preferably equal to or more than 20 percent by weight, and most preferably 25 percent by weight) of all the (meth)acrylate compounds contained in the mixture. The upper limit of the content of the di(meth)acrylate compound in the mixture is typically 99 percent by weight, preferably 90 percent by weight, more preferably 60 percent by weight, particularly preferably 50 percent by weight, most preferably 40 percent by weight, and still more preferably 35 percent by weight. In the mixture, the total content of the mono(meth)acrylate compound and the di(meth)acrylate compound may be typically equal to or more than 50 percent by weight, preferably equal to or more than 70 percent by weight, particularly preferably equal to or more than 80 percent by weight, and most preferably from 90 percent by weight to 100 percent by weight, based on the total weight of the mixture. Formulae (1′) and (4) are expressed as follows:

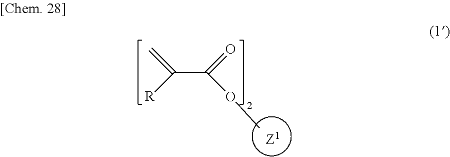

where Ring Z¹ represents a tricyclo[5.2.1.0^{2,6}]decane ring; and R is selected from a hydrogen atom and a methyl group,

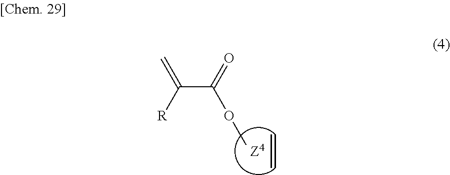

where Ring Z⁴ is a ring corresponding to a tricyclo[5.2.1.0^{2,6}]decane ring, except for containing one carbon-carbon double bond instead of one of carbon-carbon single bonds constituting the tricyclo[5.2.1.0^{2,6}]decane ring; and R is selected from a hydrogen atom and a methyl group.

The mixture including the mono(meth)acrylate compound and the di(meth)acrylate compound may contain the mono(meth)acrylate compound in a content of typically equal to or more than 10 percent by weight, preferably equal to or more than 20 percent by weight, particularly preferably equal to or more than 30 percent by weigh, and most preferably 40 percent by weight) of all the (meth)acrylate compounds contained in the mixture. The upper limit of the content of the mono(meth)acrylate compound is typically 90 percent by weight, preferably 80 percent by weight, more preferably 70 percent by weight, furthermore preferably 65 percent by weight, particularly preferably 60 percent by weight, and most preferably 55 percent by weight.

In the mixture including the mono(meth)acrylate compound and the di(meth)acrylate compound, the ratio (weight ratio) in content of the mono(meth)acrylate compound to the di(meth)acrylate compound is typically from 10:90 to 80:20, preferably from 30:70 to 80:20, more preferably from 40:60 to 70:30, particularly preferably from 45:55 to 70:30, and most preferably from 55:45 to 65:35.

The mixture including the mono(meth)acrylate compound and the di(meth)acrylate compound may be produced typically by allowing 1 mole of tricyclo[5.2.1.0^{2,6}]deca-3,8-diene (i.e., dicyclopentadiene) to react with 1.1 moles or more (preferably 1.1 to 20 moles, particularly preferably 2 to 10 moles) of (meth)acrylic acid in the presence of a Lewis acid catalyst such as boron trifluoride-diethyl ether complex.

Since having the configurations, the multifunctional (meth)acrylate compounds according to the present invention, which are represented by Formula (1), and the mixtures according to the present invention can be cured upon application typically of an ultraviolet ray and can form cured products that offer high hardness and high elastic moduli, have low plastic deformation work, and have resistance to deformation. The compounds and mixtures are therefore useful as raw materials for high-hardness hard-coating agents.

The cured products of the multifunctional (meth)acrylate compounds according to the present invention, represented by Formula (1), and the cured products of the mixtures according to the present invention have excellent heat resistance and have glass transition temperatures (Tg) of equal to or higher than 230° C., and preferably 240° C. to 270° C. In addition, the cured products have high hardness, high elastic moduli, and low plastic deformation works and have resistance to deformation. The cured products have Martens hardnesses of typically equal to or more than 300 N/mm² and preferably equal to or more than 350 N/mm² and have indentation hardnesses of typically equal to or more than 420 N/mm², preferably equal to or more than 500 N/mm², and particularly preferably equal to or more than 550 N/mm². The cured products have elastic moduli of typically equal to or more than 7000 N/mm², preferably equal to or more than 7500 N/mm², and particularly preferably equal to or more than 7700 N/mm². The cured products have plastic deformation works of typically equal to or less than 3.3×10⁻¹¹ N·m, preferably equal to or less than 3.0×10⁻¹¹ N·m, particularly preferably equal to or less than 2.5×10⁻¹¹ N·m.

The high-hardness hard-coating agent (coating agent for high-hardness hard coat) according to the present invention contains a radical polymerization initiator and, as a radically polymerizable compound or compounds, at least one selected from the multifunctional (meth)acrylate compounds represented by Formula (1) and the mixtures. The high-hardness hard-coating agent contains the radical polymerization initiator in a content of typically 0.1 to 10 parts by weight per 100 parts by weight of the radically polymerizable compound(s). In addition to the components, the high-hardness hard-coating agent according to the present invention may further contain one or more other components within ranges not adversely affecting the advantageous effects of the present invention.

Since having the configuration, the high-hardness hard-coating agent according to the present invention can give a cured product that has excellent heat resistance, offers high hardness and a high elastic modulus, has a low plastic deformation work, and has resistance to deformation. This allows the high-hardness hard-coating agent to be advantageously usable typically for high-hardness hard coat films for portable communication devices or portable information devices; and optical sheets.

### EXAMPLES

The present invention will be illustrated in further detail with reference to several examples below. It should be noted, however, that the examples are by no means intended to limit the scope of the present invention. The purities of reaction products were measured by gas chromatography.

### Example 1

Into a 300-mL four-necked flask equipped with a stirrer, a thermometer, a condenser, a dropping funnel, and an air (containing 8% oxygen) bubbling line, 0.6588 g (5.98 mmol) of hydroquinone, 48.84 g (567 mmol) of methacrylic acid, and 2.36 g (16.65 mmol) of boron trifluoride-diethyl ether complex were charged.

Into the 200-mL dropping funnel, 50 g (378.19 mmol) of dicyclopentadiene and 48.84 g (567.28 mmol) of methacrylic acid were charged and mixed with each other to give a solution.

After nitrogen purge, bubbling of air (containing 8% oxygen) at a rate of 300 mL/min was started, followed by heating to 70° C. The solution in the dropping funnel was dropped at 70° C. over 20 minutes. The dropping start time was defined as a reaction start time, and, one hour after the reaction start, the hot-water bath was cooled to cool the reaction mixture down to room temperature.

The reaction mixture was combined with 68 g of n-heptane and 134.78 g of 5% sodium carbonate aqueous solution and subjected to an extraction operation using a separatory funnel. After the lower layer (aqueous layer) was drawn off, 134.78 g of water and 33.69 g of acetonitrile were added, followed by extraction operation. After the lower layer was drawn off again, 134.78 g of water were added, followed by extraction operation. After the lower layer was drawn off still again, the upper layer (organic layer) was taken out. The organic layer was roughly concentrated on an evaporator at about 70° C. and about 20 mmHg to distill off n-heptane and acetonitrile from the organic layer.

In addition, the crude concentrated liquid was subjected to simple distillation (at a bath temperature of 180° C.) with gradual pressure reduction to distill off methacrylic acid as an initial distillation (first fraction). At the time when distilling of methacrylic acid terminated, the pressure was further reduced (70 to 100 Pa) and the distilling line temperature was set to 100° C. or higher. Then, dicyclopentenyl monomethacrylate began distilling. While waiting until the distilling terminated, 66 g of liquid dicyclopentenyl monomethacrylate was obtained. This was a mixture of stereoisomers and regioisomers in a compound represented by Formula (E1) below. The dicyclopentenyl monomethacrylate was obtained with a purity of 96% in a yield of 80%.

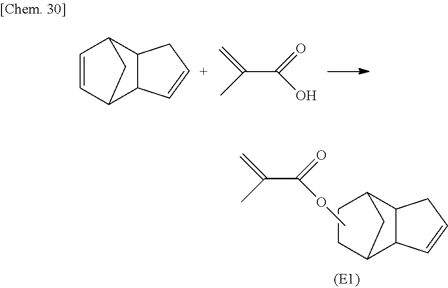

Into a 500-mL four-necked flask equipped with a stirrer, a thermometer, a condenser, a dropping funnel, and a nitrogen vent line, 40 g (183.23 mmol) of the prepared dicyclopentenyl monomethacrylate and 60 g of ethyl acetate were charged.

Separately, 63.24 g (256.53 mmol) of m-chloroperbenzoic acid (hereinafter also referred to as “mCPBA”) and 208.70 g of ethyl acetate were placed in an Erlenmeyer flask, stirred for dissolution, and yielded an mCPBA solution in ethyl acetate.

The prepared mCPBA solution in ethyl acetate was charged into the dropping funnel, the reaction system was purged with nitrogen, and the reaction system was adjusted so as to allow the solution temperature to be 20° C. While maintaining the solution temperature at 20° C., dropping of the mCPBA solution in ethyl acetate was started, and this was defined as a reaction start. The mCPBA solution in ethyl acetate was dropped over 20 minutes, and the reaction was performed for 7 hours after the dropping start.

After the completion of the reaction, the reaction mixture was combined with 186.97 g of n-heptane and 240 g of 5% sodium thiosulfate aqueous solution and was subjected to a quenching operation in a separatory funnel.

Specifically, the mixture was subjected to an alkaline quenching operation two times. In the alkali quenching operation, after the lower layer (aqueous layer) was drawn off, 240 g of 5% sodium hydroxide aqueous solution was added, followed by extraction.

The mixture was further subjected, two times, to an operation of drawing off the lower layer and thereafter adding 185.97 g of water to perform water washing.

The upper layer liquid after extraction was taken out, concentrated on an evaporator at 40° C. or lower, and yielded 76 g of a liquid epoxidized dicyclopentanyl monomethacrylate. This was a mixture of stereoisomers and regioisomers in a compound represented by Formula (E2) below. The epoxidized dicyclopentanyl monomethacrylate was obtained with a purity of 94% in a yield of 86%.

The structure of the epoxidized dicyclopentanyl monomethacrylate was identified by ¹H-NMR analysis and mass spectrometry (MS).

¹H-NMR (500 MHz, CDCl₃): δ=6.06 (d, 1H), 5.54 (d, 1H), 4.68 (m, 1H), 3.47 (d, 1H), 3.28 (d, 1H), 1.34-2.37 (m, 13H)

Mass (EI, M⁺): 234, 148, 69

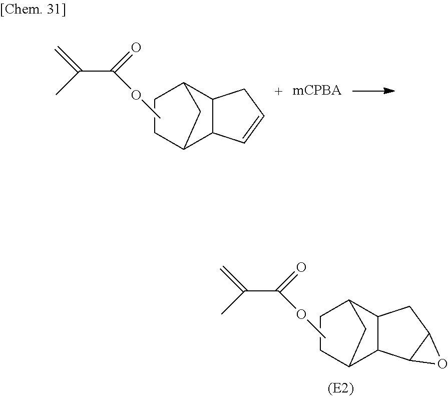

Into a 300-mL four-necked flask equipped with a stirrer, a thermometer, a condenser, a dropping funnel, and an air (containing 8% oxygen) bubbling line, 1.8697 g (16.98 mmol) of hydroquinone, 6.30 g (50.76 mmol) of potassium methacrylate, and 73.49 g (853 mmol) of methacrylic acid were charged.

Into the dropping funnel, 40 g (170.72 mmol) of the epoxidized dicyclopentanyl monomethacrylate and 73.49 g (853.91 mmol) of methacrylic acid were charged.

After the reaction system was purged with nitrogen, bubbling of 300 mL of air (containing 8% oxygen) was started, followed by heating to 120° C. on an oil bath. Dropping was started at 120° C., and this was defined as a reaction start. The dropping was performed over 10 minutes. The reaction was kept on for 12 hours and yielded a reaction mixture including hydroxydicyclopentanyl dimethacrylate and dicyclopentanyl trimethacrylate, where the hydroxydicyclopentanyl dimethacrylate was a mixture of stereoisomers and regioisomers in a compound represented by Formula (E3), and the dicyclopentanyl trimethacrylate was a mixture of stereoisomers and regioisomers in a compound represented by Formula (E4). Hydroxydicyclopentanyl dimethacrylate and dicyclopentanyl trimethacrylate were yielded respectively in yields of 83.5% and 1%, where the percentages were determined as GC area percentages by area percentage analysis using gas chromatography. The conversion from the epoxidized dicyclopentanyl monomethacrylate was 93%.

The structure of hydroxydicyclopentanyl dimethacrylate was identified by ¹H-NMR analysis and mass spectrometry (MS).

¹H-NMR (500 MHz, CDCl₃): δ=6.12 (d, 1H), 6.07 (d, 1H), 5.60 (d, 1H), 5.53 (d, 1H), 4.82-4.87 (m, 1H), 4.63-4.64 (m, 1H), 3.70-3.74 (m, 1H), 3.20 (br, 1H), 1.17-2.44 (m, 16H)

Mass (EI, M⁺): 234, 148, 69

The structure of dicyclopentanyl trimethacrylate was identified by mass spectrometry (MS).

Mass (EI, M⁺): 389 (M+1), 303, 217, 69

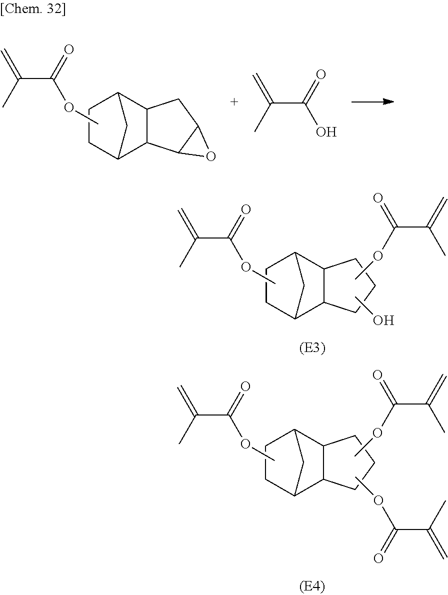

In a separatory funnel, 190 g of the reaction mixture and 190 g of saturated sodium chloride aqueous solution were placed, followed by extraction at 50° C. for 15 minutes. The lower layer liquid was drawn off, and 190 g of water and 190 g of n-heptane were added, followed by extraction at room temperature. The lower layer liquid was drawn off, 190 g of water were added, followed by extraction again, and the resulting lower layer liquid was drawn off. The upper layer liquid was combined with a solution of 0.1914 g of hydroquinone in 2.4 g of acetone, concentrated on an evaporator at 100° C. under full vacuum to distill off the solvent and methacrylic acid, and yielded 61 g of a brown concentrate containing 71 percent by weight of non-volatile components (hydroxydicyclopentanyl dimethacrylate and dicyclopentanyl trimethacrylate).

The above operation was repeated four times, and the concentrates were combined.

The resulting concentrate (189.6 g) was subjected to column purification using a silica gel and an eluent (developing solvent) (1:2 mixture of ethyl acetate and hexane). This yielded 40 g of a liquid component and 30 g of a solid component.

The liquid component and the solid component obtained via column purification were independently analyzed using gas chromatography [trade name GC-2010 (supplied by Shimadzu Corporation), column: DB-1]. The results are collectively shown in the table below. The numerical values in Table 1 are GC area percentages.

TABLE 1

| | Dimethacrylate | | | | | | |
|---|---|---|---|---|---|---|---|
| | GC retention time | | | | | Dimethacrylate | |
| | 33.8 min. | 34.4 min. | 34.6 min. | 35.3 min. | 35.6 min. | Total | Trimethacrylate |
| Solid component | 1.86 | 5.76 | 3.42 | 6.20 | 77.65 | 94.48 | 2.08 |
| Liquid component | 0.79 | 19.38 | 10.41 | 19.76 | 36.67 | 86.55 | 4.69 |
| MS molecular | 320 | 320 | 320 | 320 | 320 | — | 388 |
| weight | | | | | | | |

*Note:
Dimethacrylate: hydroxydicyclopentanyl dimethacrylate
Trimethacrylate: dicyclopentanyl trimethacrylate

### Example 2

There was used the liquid component which was obtained via the column purification and contained 86.55 GC area percentage of hydroxydicyclopentanyl dimethacrylate (“dimethacrylate”) and 4.69 GC area percentage of dicyclopentanyl trimethacrylate (“trimethacrylate”). In the liquid component, the ratio in content (weight percent) of the dimethacrylate to the trimethacrylate was 94.8:5.2. Using the liquid component, a curable composition was prepared according to the formulation given in Table 2. A coating (having a wet thickness of 40 μm) was formed from the curable composition using a bar coater (#8), irradiated with an ultraviolet ray [2 kW, 2.25 m/min., one pass (450 mW/cm², 1000 mJ/cm²)] in a nitrogen atmosphere, and yielded a cured product. The ultraviolet ray irradiation was performed using a UV irradiation system (product number ECS-401GX, supplied by Eye Graphics Co., Ltd.).

The resulting cured product was examined to measure a glass transition temperature (Tg in degree Celsius (° C.)) using a pendulum rheometer (DDV).

In addition the cured product was examined to measure a Martens hardness (N/mm²), an indentation hardness (N/mm²), an elastic modulus (N/mm²), and a plastic deformation work (N·m) using a micro-hardness tester.

### Comparative Examples 1 and 2

The procedure of Example 2 was performed, except for preparing the curable composition so as to have the formulation given in Table 2.

TABLE 2

| | | | Compar- | Compara- |
|---|---|---|---|---|
| | | | ative | ative |
| | | Example 2 | Example 1 | Example 2 |
| Curable | Hydroxydicyclopentanyl | 5 | — | — |
| compo- | dimethacrylate + | | | |
| sition | dicyclopentanyl | | | |
| | trimethacrylate | | | |
| | Tripropylene glycol | 5 | 10 | 5 |
| | diacrylate | | | |
| | Tricyclodecanedimethanol | — | — | 5 |
| | diacrylate | | | |
| | 5% Irg. 184 | 0.5 | 0.5 | 0.5 |

| | | | |
|---|---|---|---|
| Tg (° C.) | 268 | 55 | 110 |
| Martens hardness (N/mm²) | 365.4 | 156.7 | 281.4 |
| indentation hardness (N/mm²) | 600 | 205.4 | 419.2 |
| Elastic modulus (N/mm²) | 7797 | 4674 | 6574 |
| Plastic deformation work (N · m) | 2.33 × 10⁻¹¹ | 5.4 × 10⁻¹¹ | 3.27 × 10⁻¹¹ |

*Note:
Tripropylene glycol diacrylate: trade name TPGDA, supplied by DAICEL-ALLNEX LTD.
Tricyclodecanedimethanol diacrylate: trade name IRR214-K, supplied by DAICEL-ALLNEX LTD.
5% Irg. 184: radical polymerization initiator, 5% diluted liquid of 1-hydroxycyclohexyl phenyl ketone, trade name IRGACURE 184, supplied by BASF Japan Ltd.

### Example 3

Into a 2-L four-necked flask equipped with a stirrer, a thermometer, a condenser, a dropping funnel, and an air (containing 6% oxygen) bubbling line, 3.18 g (28.88 mmol) of hydroquinone, 0.1 g (0.5 mmol) of phenothiazine, 817.6 g (11.3 mol) of acrylic acid, and 40.26 g (283.6 mmol) of boron trifluoride-diethyl ether complex were charged.

Into the 1-L dropping funnel, 500 g (3.78 mol) of dicyclopentadiene and 272.5 g (3.78 mol) of acrylic acid were charged and mixed with each other to give a solution.

After nitrogen purge, bubbling of air (containing 6% oxygen) at a rate of 750 mL/min was started, followed by heating to 70° C. The solution in the dropping funnel was dropped at 70° C. over 30 minutes. The dropping start time was defined as a reaction start time. After the dropping, the reaction temperature was raised up to 100° C. The reaction was continued for 6 hours after the dropping start, followed by cooling to cool the reaction mixture down to room temperature.

The reaction mixture was combined with 408 g of n-heptane and 1663 g of 5% sodium carbonate aqueous solution and subjected to an extraction operation in a 5-L separable flask. After the lower layer (aqueous layer) was drawn off, 1663 g of water were added, followed by extraction operation. After the lower layer was drawn off again, 1663 g of water were added, followed by extraction operation. Distilling off of the solvent n-heptane at 50° C. from the organic layer after extraction yielded 700 g of a transparent, brown mixture (1). The mixture (1) included 33 percent by weight of dicyclopentanyl diacrylate (hereinafter also referred to as “DCPDA”), 43 percent by weight of dicyclopentenyl monoacrylate (hereinafter also referred to as “DCPA”), and 23 percent by weight of a high-boiling component.

An aliquot (150 g) of the obtained mixture (1) was subjected to acetonitrile extraction to give a concentrate, and the obtained concentrate was combined with 300 g of n-heptane, 23 g of acetonitrile, and 3 g of water, followed by extraction using a separatory funnel. After the lower layer liquid (acetonitrile layer) was drawn off, the upper layer liquid was combined again with 10 g of acetonitrile, followed by extraction. This extraction operation was performed two times. Thus, a total of three acetonitrile extractions was performed, and the drawn-off lower layer liquids were collected and combined with about 100 g of n-heptane, followed by backward extraction (recovery from the lower layer liquids). The recovered upper layer liquid (n-heptane layer) and the extracted upper layer liquid were combined with each other, from which the solvent n-heptane was distilled off on an evaporator at 50° C. in a full vacuum. This yielded 116 g of a mixture (2). The mixture (2) included 29.6 percent by weight of DCPDA, 49.8 percent by weight of DCPA, and 20.6 percent by weight of a high-boiling component.

Ten (10) parts by weight of the obtained mixture (2) were combined with 0.5 part by weight of Irg. 184 and yielded a curable composition.

### Example 4

Into a 1-L four-necked flask equipped with a stirrer, a thermometer, a condenser, a dropping funnel, and an air (containing 6% oxygen) bubbling line, 1.27 g (11.55 mmol) of hydroquinone, 0.04 g (0.2 mmol) of phenothiazine, 327.0 g (4.6 mol) of acrylic acid, and 16.10 g (113.6 mmol) of boron trifluoride-diethyl ether complex were charged.

Into the 500-mL dropping funnel, 200 g (1.51 mol) of dicyclopentadiene and 109.0 g (1.51 mol) of acrylic acid were charged, and mixed with each other to give a solution.

After nitrogen purge, bubbling of air (containing 6% oxygen) at a rate of 320 mL/min was started, followed by heating to 70° C. The solution in the dropping funnel was dropped at 70° C. over 30 minutes. The dropping start time was defined as a reaction start time. After the dropping, the reaction temperature was raised up to 100° C. The reaction was continued for 2 hours after the dropping start, followed by cooling to cool the reaction mixture down to room temperature.

The reaction mixture was combined with 490 g of n-heptane and 654 g of 5% sodium carbonate aqueous solution and subjected to an extraction operation using a 3-L separatory funnel. After the lower layer (aqueous layer) was drawn off, 654 g of water were added, followed by extraction operation. After the lower layer was drawn off again, 654 g of water were added, followed by extraction operation. Distilling off of the solvent n-heptane on an evaporator at 50° C. from the organic layer after extraction yielded 291 g of a transparent, brown mixture (3). The mixture (3) included 24 percent by weight of DCPDA, 66 percent by weight of DCPA, and 10 percent by weight of a high-boiling component.

An aliquot (130 g) of the obtained mixture (3) was combined with 26 g of a high-boiling solvent (trade name GR-175, supplied by MATSUMURA OIL Co., Ltd.) and 0.065 g of phenothiazine, to give a uniform mixture. The resulting mixture was subjected to thin-film distillation at 140° C. and 120 Pa using a wiped film evaporator (WFE) (thin-film evaporator, supplied by Asahi Glassplant Inc. and Shinko Pantec Co., Ltd.) and yielded 92 g of a colorless mixture (4). Based on the GC (supplied by Shimadzu Corporation) analysis, the mixture (4) was found to include 21 percent by weight of DCPDA and 78 percent by weight of DCPA.

Ten (10) parts by weight of the obtained mixture (4) were combined with 0.5 part by weight of Irg. 184 and yielded a curable composition.

### Example 5

An aliquot (200 g) of the mixture (1) obtained in Example 3 was combined with 40 g of a high-boiling solvent (trade name GR-175, supplied by MATSUMURA OIL Co., Ltd.) and 0.1 g of phenothiazine, subjected to thin-film distillation at 145° C. and 100 Pa, and yielded 110 g of a colorless mixture (5). Based on a GC analysis, the mixture (5) was found to include 38 percent by weight of DCPDA and 62 percent by weight of DCPA.

Ten (10) parts by weight of the obtained mixture (5) were combined with 0.5 part by weight of Irg. 184 and yielded a curable composition.

### Example 6

An aliquot (130 g) of the mixture (4) obtained in Example 4 was combined with 650 of n-heptane to give a solution. This was further combined with 13 g of powdery activated carbon, followed by stirring at room temperature for 30 minutes. The resulting mixture was subjected to pressure filtration to remove the activated carbon therefrom. Such activated carbon treatment by a similar procedure was repeated a total of three times. Distilling off of the solvent n-heptane on an evaporator at 50° C. yielded 101 g of a slightly yellow mixture (6). Based on a GC analysis, the mixture (6) was found to include 21 percent by weight of DCPDA and 75 percent by weight of DCPA.

Ten (10) parts by weight of the obtained mixture (6) was combined with 0.5 part by weight of Irg. 184 and yielded a curable composition.

### Example 7

An aliquot (21 g) of the mixture (1) obtained in Example 3 was subjected to silica gel column purification (using 1000 g of silica gel and an eluent containing 95% of n-heptane and 5% of ethyl acetate). DCPA and DCPDA were detected in this order. The DCPDA-containing fraction was concentrated and yielded a slightly yellow mixture (7). The mixture (7) included 90 percent by weight of DCPDA and 10 percent by weight of DCPA.

Ten (10) parts by weight of the obtained concentrate (7) was combined with 0.5 part by weight of Irg. 184 and yielded a curable composition.

### Example 8

The mixture (7) obtained in Example 7 was mixed with the mixture (1) obtained in Example 3 and yielded a mixture (8). The mixture (8) included 51 percent by weight of DCPDA and 49 percent by weight of DCPA.

Ten (10) parts by weight of the obtained mixture (8) was combined with 0.5 part by weight of Irg. 184 and yielded a curable composition.

Evaluations

The curable compositions obtained in Examples 3 to 8, and curable compositions having formulations (in part by weight) given in Table 3 as Comparative Examples 3 and 4 were used to form coatings (having a wet thickness of 40 μm). The coatings were irradiated with an ultraviolet ray in a nitrogen atmosphere [2 kW, 2.25 m/min., one pass (450 mW/cm², 1000 mJ/cm²)] and yielded cured products. The obtained cured products were evaluated by a similar procedure to Example 2. Comparative Example 4 employed a product under the trade name of FS-511AS (supplied by Hitachi Chemical Company, Ltd.) as dicyclopentenyl monoacrylate.

TABLE 3

| | | | | | | | | Comparative | Comparative |
|---|---|---|---|---|---|---|---|---|---|
| | | Example 3 | Example 4 | Example 5 | Example 6 | Example 7 | Example 8 | Example 3 | Example 4 |
| Curable | Dicyclopentenyl | 49.8 | 78 | 62 | 75 | 10 | 49 | — | 100 |
| composition | monoacrylate | | | | | | | | |
| | Dicyclopentanyl | 29.6 | 21 | 38 | 21 | 90 | 51 | — | — |
| | diacrylate | | | | | | | | |
| | Tricyclodecanedi- | — | — | — | — | — | — | 100 | — |
| | methanol | | | | | | | | |
| | diacrylate | | | | | | | | |
| | High-boiling | 20.6 | 1 | 0 | 4 | 0 | 0 | — | — |
| | component | | | | | | | | |
| | 5% Irg. 184 | 5 | 5 | 5 | 5 | 5 | 5 | 5 | 5 |

| Martens hardness (N/mm²) | | 400.8 | 331 | 348 | 340 | 393 | 380 | 262 | 330.3 |
|---|---|---|---|---|---|---|---|---|---|
| Indentation hardness (N/mm²) | | 630.4 | 517 | 546 | 541 | 602 | 575 | 364 | 491 |
| Elastic modulus (N/mm²) | | 8266 | 7384 | 7981 | 7656 | 8440 | 8430 | 7075 | 7336 |
| Plastic deformation work | | 3.15 × 10⁻¹¹ | 3.26 × 10⁻¹¹ | 3.10 × 10⁻¹¹ | 3.20 × 10⁻¹¹ | 3.263 × 10⁻¹¹ | 3.29 × 10⁻¹¹ | 4.09 × 10⁻¹¹ | 3.91 × 10⁻¹¹ |
| (N · m) | | | | | | | | | |
| Martens | Relative to | 153 | 126 | 133 | 130 | 150 | 145 | 100 | — |
| hardness | IRR214-K | | | | | | | | |
| (N/mm²) | Relative to | 121 | 100 | 105 | 103 | 119 | 115 | — | 100 |
| | FS-511AS | | | | | | | | |
| Indentation | Relative to | 173 | 142 | 150 | 149 | 165 | 158 | 100 | — |
| hardness | IRR214-K | | | | | | | | |
| (N/mm²) | Relative to | 128 | 105 | 111 | 110 | 123 | 117 | — | 100 |
| | FS-511AS | | | | | | | | |
| Elastic | Relative to | 117 | 104 | 113 | 108 | 119 | 119 | 100 | — |
| modulus | IRR214-K | | | | | | | | |
| (N/mm²) | Relative to | 113 | 101 | 109 | 104 | 115 | 115 | — | 100 |
| | FS-511AS | | | | | | | | |
| Plastic | Relative to | 123 | 120 | 124 | 122 | 120 | 119 | 100 | — |
| deformation | IRR214-K | | | | | | | | |
| work (N · m) | Relative to | 119 | 117 | 121 | 118 | 117 | 116 | — | 100 |
| | FS-511AS | | | | | | | | |

*Note:
Tricyclodecanedimethanol diacrylate: product under the trade name of IRR214-K, supplied by DAICEL-ALLNEX LTD.
5% Irg. 184: radical polymerization initiator, 5% diluted liquid of 1-hydroxycyclohexyl phenyl ketone, trade name IRGACURE 184, supplied by BASF Japan Ltd.

### INDUSTRIAL APPLICABILITY

The multifunctional (meth)acrylate compounds and mixtures according to the present invention have excellent curability (polymerizability) and can form polymers or cured products having high hardness and high elastic moduli and have excellent strength. The multifunctional (meth)acrylate compounds and mixtures are thereby useful typically as glass-substitute materials.

Of the multifunctional (meth)acrylate compounds according to the present invention, hydroxy-containing compounds each have a hydroxy group which can be easily converted into a specific chemical group. The hydroxy-containing compounds, when undergone the conversion, can be used as raw materials (i.e., functional monomers) for resins having a specific function or functions (i.e., functional resins). Accordingly, the multifunctional (meth)acrylate compounds according to the present invention are also useful as precursors for such functional monomers.

1. A multifunctional (meth)acrylate compound represented by Formula (1): 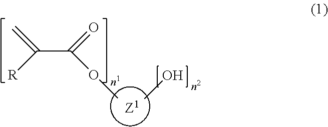
wherein Ring Z¹ represents a tricyclo[5.2.1.0^{2,6}]decane ring; R is, independently in each occurrence, selected from a hydrogen atom and a methyl group; and n¹ and n² meet a condition that n¹ is 2 or 3 and n² is 1, or a condition that n¹ is 3 or 4 and n² is 0.

2. A mixture comprising
a multifunctional (meth)acrylate compound represented by Formula (1): 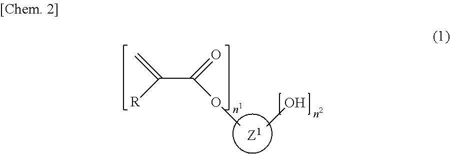
wherein Ring Z¹ represents a tricyclo[5.2.1.0^{2,6}]decane ring; R is, independently in each occurrence, selected from a hydrogen atom and a methyl group; and n¹ and n² meet a condition that n¹ is 2 or 3 and n² is 1, or a condition that n¹ is 3 or 4 and n² is 0.

3. The mixture according to claim 2,
wherein the mixture is obtained by allowing a compound represented by Formula (2) or a compound represented by Formula (3) to react with (meth)acrylic acid, Formula (2) and Formula (3) expressed as follows: 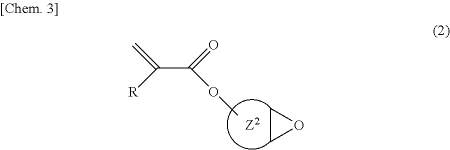
wherein Ring Z² is a ring corresponding to a tricyclo[5.2.1.0^{2,6}]decane ring, except for having one epoxy group, the epoxy group containing one oxygen atom bonded in a triangular arrangement to two adjacent carbon atoms constituting the tricyclo[5.2.1.0^{2,6}]decane ring; and R is selected from a hydrogen atom and a methyl group, 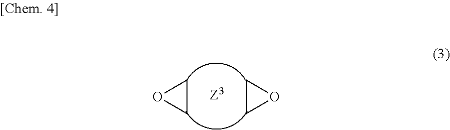
wherein Ring Z³ is a ring corresponding to a tricyclo[5.2.1.0^{2,6}]decane ring, except for having two epoxy groups, the two epoxy groups each independently containing one oxygen atom bonded in a triangular arrangement to two adjacent carbon atoms constituting the tricyclo[5.2.1.0^{2,6}]decane ring.

4. A mixture of (meth)acrylate compounds comprising:
a di(meth)acrylate compound represented by Formula (1′); and
a mono(meth)acrylate compound represented by Formula (4),
the mixture comprising the di(meth)acrylate compound in a proportion of equal to or more than 10 percent by weight of all the (meth)acrylate compounds contained in the mixture, Formula (1′) and Formula (4) expressed as follows: 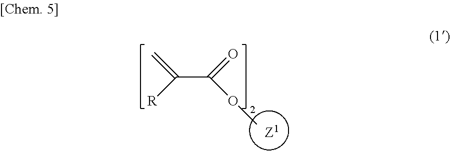
wherein Ring Z¹ represents a tricyclo[5.2.1.0^{2,6}]decane ring; and R is, independently in each occurrence, selected from a hydrogen atom and a methyl group, 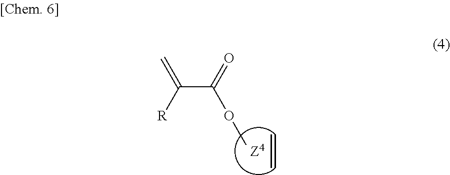
wherein Ring Z⁴ is a ring corresponding to a tricyclo[5.2.1.0^{2,6}]decane ring, except for containing one carbon-carbon double bond instead of one of carbon-carbon single bonds constituting the tricyclo[5.2.1.0^{2,6}]decane ring; and R is selected from a hydrogen atom and a methyl group.

5. The mixture according to claim 4,
wherein the mixture is obtained by allowing 1 mole of tricyclo[5.2.1.0^{2,6}]deca-3,8-diene to react with 1.1 moles or more of (meth)acrylic acid in the presence of a Lewis acid catalyst.

6. The mixture according to claim 5,
wherein the Lewis acid catalyst is boron trifluoride-diethyl ether complex.

7. A method for producing a multifunctional (meth)acrylate compound, the method comprising
allowing a compound represented by Formula (2) or a compound represented by Formula (3) to react with (meth)acrylic acid to yield a multifunctional (meth)acrylate compound represented by Formula (1), Formula (2), Formula (3), and Formula (1) expressed as follows: 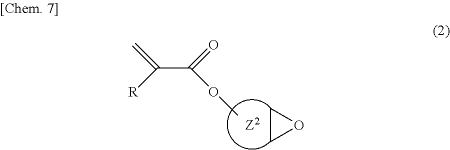
wherein Ring Z² is a ring corresponding to a tricyclo[5.2.1.0^{2,6}]decane ring, except for having one epoxy group, the epoxy group containing one oxygen atom bonded in a triangular arrangement to two adjacent carbon atoms constituting the tricyclo[5.2.1.0^{2,6}]decane ring; and R is selected from a hydrogen atom and a methyl group, 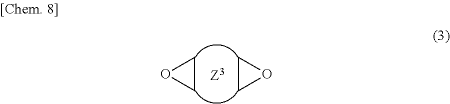
wherein Ring Z³ is a ring corresponding to a tricyclo[5.2.1.0^{2,6}]decane ring, except for having two epoxy groups, the two epoxy groups each independently containing one oxygen atom bonded in a triangular arrangement to two adjacent carbon atoms constituting the tricyclo[5.2.1.0^{2,6}]decane ring, 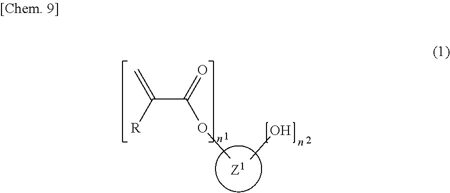
wherein Ring Z¹ represents a tricyclo[5.2.1.0^{2,6}]decane ring; n¹ n and n² meet a condition that n¹ is 2 or 3 and n² is 1, or a condition that n¹ is 3 or 4 and n² is 0; and R is, independently in each occurrence, as defined above.

8. The method according to claim 7 for producing a multifunctional (meth)acrylate compound,
wherein a compound represented by Formula (4) is allowed to react with a peracid to yield a compound represented by Formula (2); and
wherein the obtained compound represented by Formula (2) is allowed to react with (meth)acrylic acid, Formula (4) and Formula (2) expressed as follows: 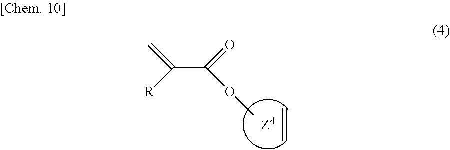
wherein Ring Z⁴ is a ring corresponding to a tricyclo[5.2.1.0^{2,6}]decane ring, except for containing one carbon-carbon double bond instead of one of carbon-carbon single bonds constituting the tricyclo[5.2.1.0^{2,6}]decane ring; and R is selected from a hydrogen atom and a methyl group, 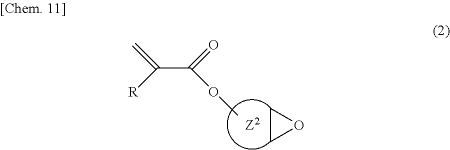
wherein Ring Z² is a ring corresponding to a tricyclo[5.2.1.0^{2,6}]decane ring, except for having one epoxy group, the epoxy group containing one oxygen atom bonded in a triangular arrangement to two adjacent carbon atoms constituting the tricyclo[5.2.1.0^{2,6}]decane ring; and R is as defined above.

9. The method according to claim 7 for producing a multifunctional (meth)acrylate compound,
wherein a compound represented by Formula (5) is allowed to react with a peracid to yield a compound represented by Formula (3); and
wherein the obtained compound represented by Formula (3) is allowed to react with (meth)acrylic acid, Formula (5) and Formula (3) expressed as follows: 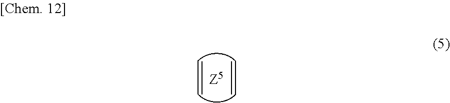
wherein Ring Z⁵ is a ring corresponding to a tricyclo[5.2.1.0^{2,6}]decane ring, except for containing two carbon-carbon double bonds instead of two carbon-carbon single bonds constituting the tricyclo[5.2.1.0^{2,6}]decane ring, 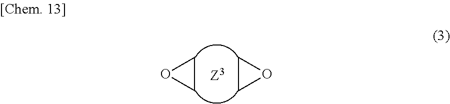
wherein Ring Z³ is a ring corresponding to a tricyclo[5.2.1.0^{2,6}]decane ring, except for having two epoxy groups, the two epoxy groups each independently containing one oxygen atom bonded in a triangular arrangement to two adjacent carbon atoms constituting the tricyclo[5.2.1.0^{2,6}]decane ring.

10. A polymer comprising
a monomer unit derived from the multifunctional (meth)acrylate compound according to claim 1.

11. A high-hardness hard-coating agent comprising
the multifunctional (meth)acrylate compound according to claim 1 or the mixture according to any one of claims 2 to 6.

12. A cured product of the high-hardness hard-coating agent according to claim 11.
